(19) United States
(12) Patent Application Publication
(10) Pub. No.: US 2026/0306531 A1
(43) Pub. Date: Oct. 8, 2026

(54) Microbiome-based GABA Cream with Chondroitin Sulfate Transdermal Delivery System

(71) Applicant: Stefania Pacini, Mesa, AZ (US)

(72) Inventor: Stefania Pacini, Mesa, AZ (US)

(73) Assignee: Stefania Pacini, Mesa, AZ (US)

(21) Appl. No.: 19/577,188

(22) Filed: Mar. 24, 2026

Related U.S. Application Data
(60) Provisional application No. 63/785,540, filed on Apr. 8, 2025.

(51) Int. Cl. A61K 31/737; A61K 9/06; A61K 31/197; A61K 35/744; A61K 35/745; A61K 35/747; A61K 36/064; A61K 47/10; A61K 47/12; A61K 47/22; A61K 47/44; A61K 47/54; A61P 25/22

(52) U.S. Cl. CPC A61K 31/737; A61K 9/06; A61K 31/197; A61K 35/744; A61K 35/745; A61K 35/747; A61K 36/064; A61K 47/10; A61K 47/12; A61K 47/22; A61K 47/44; A61K 47/544; A61P 25/22

(57) ABSTRACT

A novel topical cream comprising Gamma-Aminobutyric Acid (GABA), microbial chondroitin sulfate to enhance GABA's effect, and fermented probiotic microorganisms, formulated with a delivery system and excipients, along with the methods for its manufacture are disclosed. This topical cream exhibit ability to provide relief from anxiety, improve sleep quality, enhance cognitive functions, and alleviate joint, muscle, and post-traumatic pain.

### CROSS-REFERENCE TO RELATED US APPLICATIONS DATA

This application claims the benefit of U.S. Provisional Patent Application No. 63/785,540. Patent Center No. 69922359. Confirmation No. 7775. Filed on Apr. 8, 2025.

### GOVERNMENT CONTRACT

Not applicable

### STATEMENT RE. FEDERALLY SPONSORED RESEARCH/DEVELOPMENT

Not applicable

### COPYRIGHT & TRADEMARK NOTICES

A portion of the disclosure of this patent document may contain material which is subject to copyright protection. This patent document may show and/or describe matter which is or may become trade dress of the owner. The copyright and trade dress owner has no objection to the facsimile reproduction by any one of the patent document or the patent disclosure, as it appears in the US Patent and Trademark Office patent files or records, but otherwise reserves all copyrights and trade dress rights whatsoever.

### TECHNICAL FIELD

The present invention lies in the technical field of topical formulations for health and wellness. More specifically, this invention concerns a novel cream comprising Gamma-Aminobutyric Acid (GABA), microbial chondroitin sulfate, and fermented probiotic microorganisms, formulated for applications in anxiety management, sleep quality enhancement, improvement of cognitive functions, and relief of joint, muscle, and post-traumatic pain

### REFERENCES CITED

### US Patent Documents

U.S. Pat. No. 9,109,243B2. James F. Jolly, Krzysztof Klimaszewski, Yuji Nakanishi, Hirotaka Matsubara, Tetsuya, Takahashi, Kyouichi Nishio. Microbial-derived chondroitin sulfate. Pub. Date: Aug. 18, 2015.
U.S. Pat. No. 9,327,105B2. Radhakrishnan Ramdas, Kartik Karri, Chidella Venkata, Krishna Mohan Sharma. Active transdermal drug delivery system and the method thereof. Pub. Date: May 3, 2016
US20210401731A1. Marco Ruggiero M. Anti-Aging Formula. Pub. Date: Dec. 30, 2021.
US20220401502A1. Marco Ruggiero M. Composition, methods and rationale for the formula of a pre-probiotic dietary supplement aimed at protecting the human body from the harmful effects of electromagnetic radiations through quantum entanglement of DNA repair genes elicited by natural superluminal particles inducing photosynthesis via Cherenkov effect. Pub. Date: Dec. 22, 2022.

### OTHER PUBLICATIONS

Corradetti R, Ruggiero M, Chiarugi V P, Pepeu G. GABA-receptor stimulation enhances norepinephrine-induced polyphosphoinositide metabolism in rat hippocampal slices. Brain Res. 1987 May 12; 411(1): 196-9. doi: 10.1016/0006-8993(87)90701-3. PMID: 3038260.
Ruggiero M, Corradetti R, Chiarugi V, Pepeu G. Phospholipase C activation induced by noradrenaline in rat hippocampal slices is potentiated by GABA-receptor stimulation. EMBO J. 1987 June; 6(6):1595-8. doi: 10.1002/j.1460-2075.1987.tb02405.x. PMID: 3038526; PMCID: PMC553529.
Ruggiero, M. (2024). Effects of a Topical Composition of GABA and Microbial Chondroitin Sulfate on Mental Calmness and Brain Function. American Journal of Immunology, 19(1), 1-10. https://doi.org/10.3844/ajisp.2024.1.10

### INVENTOR'S EXPERTISE

Dr. Stefania Pacini, MD, PhD, has accrued substantial research experience in the following areas: Transdermal delivery of bioactive substances. Pre- and probiotics. Microbiome medicine. Fermentation processes. Such an expertise is documented in the peer-reviewed publications listed below.
1. Pacini S, Punzi T, Gulisano M, Cecchi F, Vannucchi S, Ruggiero M. Transdermal delivery of heparin using pulsed current iontophoresis. Pharm Res. 2006 January; 23(1):114-20. doi: 10.1007/s11095-005-8923-z. Epub 2006 Dec. 21. PMID: 16362453.
2. Pacini S, Gulisano M, Punzi T, Ruggiero M. Transdermal delivery of Clostridium botulinum toxin type A by pulsed current iontophoresis. J Am Acad Dermatol. 2007 December; 57(6):1097-9. doi: 10.1016/j.jaad.2007.08.037. PMID: 18021859.
3. Pacini S and Ruggiero M Description of a Novel Probiotic Concept: Implications for the Modulation of the Immune System. American Journal of Immunology 2017, 13 (2): 107.113 DOI: 10.3844/ajisp.2017.107.113
4. Blythe, J, Ruggiero M and Pacini S Case report: intermittent fasting and probiotic yogurt consumption are associated with reduction of serum alpha-N-acetylgalactosaminidase and increased urinary excretion of lipophilic toxicants. Madridge J Immunology 2017 1(1): 23-27.
5. Nicola Antonucci, Stefania Pacini and Marco Ruggiero. Use of an Extremely Biodiverse Probiotic and a Supplement Based on Microbial Chondroitin Sulfate is Associated with a Significant Decrease of Serum Free Kappa Light Chains as well as a Trend Toward Normalization of Kappa/Lambda Ratio and of Plasma Cell Bone Marrow Infiltration in a Case of Multiple Myeloma. American Journal of Immunology 2019. DOI: 10.3844/ajisp.2019.
6. Ibne Raihan Zunaid, Stefania Pacini and Marco Ruggiero. Significance of hydrophobic and charged sequence similarities in sodium-bile acid cotransporter and vitamin D-binding protein macrophage activating factor. bioRxiv doi: https://doi.org/10.1101/2020.03.03.975524.
7. Marco Ruggiero and Stefania Pacini. Fermentation of hemp seed proteins leads to formation of peptides that share sequence similarity with human vitamin D-binding protein. Med Res Innov, 2020 doi: 10.15761/MRI.1000170. Volume 4:1-3.
8. Pacini S, Ruggiero M. Natural Plasmids in a Swiss Fermented Milk and Colostrum Product assessed by Microbiome Array. Madridge J Immunol. 2019; 3(2): 100-108. doi: 10.18689/mjim-1000123.
9. Carter, M., Pacini, S. & Ruggiero, M. (2020). Consumption of an Extremely Biodiverse Probiotic and a Supplement based on Microbial Chondroitin Sulfate is Associated with Very Low Serum Alpha-N-acetylgalactosaminidase (Nagalase) Activity and Decrease of C-reactive Protein Values. American Journal of Immunology, 16(1), 8-18. https://doi.org/10.3844/ajisp.2020.8.18
10. Pacini S, Ruggiero M. Phage composition of a fermented milk and colostrum product assessed by microbiome array; putative role of open reading frames in reference to cell signaling and neurological development. J Neurol Stroke. 2020; 10(2):80-90. DOI: 10.15406/jnsk.2020.10.00416

### BACKGROUND OF THE INVENTION

Anxiety, sleep disorders, and the desire to enhance certain brain functions like meditation are common concerns for many individuals. Traditional approaches to address these issues often involve systemic interventions, such as oral or injectable medications. While these methods can be effective, they may also be associated with undesirable systemic side effects.

Gamma-Aminobutyric Acid (GABA) is a key inhibitory neurotransmitter in the central nervous system known for its calming and relaxing effects, making it a potential therapeutic agent for anxiety and sleep-related issues. However, the effective delivery of GABA, particularly through non-invasive topical application to target tissues, has presented challenges, primarily due to limitations in transdermal absorption.

To overcome these challenges, the present invention introduces a novel topical composition combining Gamma-Aminobutyric Acid (GABA) with microbial chondroitin sulfate and probiotic microorganisms. Probiotics, when applied topically, are known to offer various benefits for skin health. They can help to balance the skin's microbiome, potentially reducing inflammation, improving the skin's barrier function, and contributing to overall skin health and resilience. Certain probiotics may also have soothing and protective effects on the skin.

The inclusion of microbial chondroitin sulfate serves a dual purpose. As previously mentioned, it is intended to act as a carrier molecule, facilitating the penetration of GABA through the skin layers to potentially reach deeper tissues and interact with GABA receptors. Additionally, chondroitin sulfate, particularly when derived from microbial sources, can offer inherent benefits to the skin. It is known for its hydrating properties, helping to retain moisture and improve skin elasticity. Some studies also suggest that chondroitin sulfate may possess anti-inflammatory properties and contribute to the structural integrity of the skin. The microbial origin of the chondroitin sulfate may also offer advantages in terms of purity and consistency, and can be a preferred source for those seeking to avoid animal-derived ingredients.

This unique combination of GABA, probiotic microorganisms, and microbial chondroitin sulfate in a topical formulation aims to provide a synergistic approach. The potential for enhanced transdermal delivery of GABA, coupled with the skin health benefits of probiotics and the hydrating and potentially anti-inflammatory effects of microbial chondroitin sulfate, offers a non-invasive alternative that may avoid the systemic side effects associated with oral or injectable GABA or other anxiolytic and hypnotic drugs.

This novel formulation represents a promising avenue for addressing common concerns related to pain management, anxiety, and sleep, while also potentially supporting practices such as meditation and the exploration of nonlocal consciousness through the modulation of GABAergic pathways, all within a beneficial topical delivery system for the skin.

### SUMMARY OF THE INVENTION

The present invention relates to a novel topical formulation designed for the enhancement of health and well-being through the synergistic combination of Gamma-Aminobutyric Acid (GABA), microbial chondroitin sulfate, and a specific blend of fermented probiotic microorganisms. A primary objective of this invention is to provide an effective transdermal delivery system that overcomes the well-known challenges of GABA's systemic bioavailability when administered orally. This is achieved by utilizing microbial chondroitin sulfate as a carrier molecule, which, owing to its low molecular weight and specific sulfation pattern, facilitates the penetration of GABA through the stratum corneum to reach localized tissues and modulate GABAergic signaling pathways. Such a mechanism allows for the promotion of mental calmness, the improvement of sleep quality, and the relief of musculoskeletal and post-traumatic pain without the risks of dependency or the systemic side effects associated with traditional pharmacological interventions.

The formulation further distinguishes itself through the inclusion of postbiotic compounds derived from the fermentation of Lactobacillus, Streptococcus thermophilus, Saccharomyces, and Bifidobacterium. These fermented ingredients support the skin's microbiome and barrier function, creating a receptive environment for the active components while offering inherent anti-inflammatory benefits. The structural integrity and efficacy of the cream are further supported by a complex of sunflower-derived phosphatidylcholine and a carefully selected blend of vegetable oils and waxes, which serve as stabilizers and penetration enhancers. By integrating these diverse elements, the invention provides a multi-faceted approach to neurological and physical wellness within a stable, non-invasive topical delivery system.

### EXAMPLES OF THE PREFERRED EMBODIMENTS

The following detailed description illustrates various embodiments of the present invention, a novel topical cream formulated to provide benefits such as pain and anxiety management, sleep quality enhancement, and promotion of brain-related functions. These embodiments highlight the specific components of the cream, including the active ingredients Gamma-Aminobutyric Acid (GABA), microbial chondroitin sulfate (which enhances GABA absorption), and a blend of fermented probiotic microorganisms (Lactobacillus ferment, Streptococcus thermophilus ferment, Saccharomyces ferment, Bifidobacterium), along with a carefully selected combination of excipients such as phosphatidylcholine, propanediol, dehydroacetic acid, benzoic acid, benzyl alcohol, tocopherol, glyceryl citrate/lactate/linoleate/oleate, water, and a vegetable oil and wax blend, each contributing to the formulation's delivery system, stability, sensory attributes, and overall therapeutic efficacy. The ingredients comprising the topical cream, according to various embodiments of the present invention, are detailed below.

In a first embodiment of the present invention, the topical cream comprises Gamma-Aminobutyric Acid (GABA) as a primary active ingredient. The rationale for the inclusion of GABA is predicated upon its well-established properties as a major inhibitory neurotransmitter within the central nervous system, playing a critical role in regulating neuronal activity, promoting a state of calm and counteracting pain. GABA is a naturally occurring amino acid known to be active not only in the brain but also in peripheral tissues including the intestines, stomach, Fallopian tubes, uterus, ovaries, testes, kidneys, urinary bladder, lungs, pancreas, liver, and the immune system, suggesting a broad range of potential biological effects. Notably, GABA is involved in the catabolism of serotonin and the synthesis of melatonin, indicating its regulatory influence on sleep and reproductive functions. While oral administration of GABA has been shown to have limited bioavailability, the present invention overcomes this limitation by employing a proprietary delivery system, as detailed elsewhere in this specification, to facilitate the effective transdermal absorption of GABA. This topical delivery allows GABA to exert its beneficial effects, such as reducing excessive brain activity and promoting calmness, without the risks of addiction, dependence, tolerance, or systemic side effects associated with other GABA-mimicking substances like alcohol, barbiturates, or benzodiazepines. Furthermore, GABA has been implicated in brain long-term potentiation, potentiation of endorphins and dopamine, modulation of serotoninergic and cannabinoidergic neurotransmission, potentiation of anandamide effects on endocannabinoid receptors, potentiation of norepinephrine effects on dopaminergic neurons, induction of nitric oxide synthesis, and inhibition of the prefrontal cortex, highlighting its diverse and significant roles in neurological function, all of which contribute to the rationale for its inclusion in the present topical formulation aimed at anxiety management, sleep quality enhancement, and the promotion of brain-related functions. The GABA utilized in this embodiment is preferably of a pharmaceutical grade suitable for topical application.

In a second embodiment of the present invention, the topical cream comprises microbial chondroitin sulfate. The chondroitin sulfate utilized in this invention is derived from microbial fermentation, offering significant advantages over traditional animal-derived chondroitin sulfate. Specifically, the microbial chondroitin sulfate in this embodiment exhibits a high degree of purity, typically around 99%, which surpasses the accepted range of 90% to 105% for animal-derived counterparts as outlined in the United States Pharmacopoeia. Furthermore, the microbial chondroitin sulfate is characterized by its homogeneous nature, presenting a consistent low molecular weight profile and a uniform sulfation pattern that can be modulated to be superimposable to that of human chondroitin sulfate. This homogeneity and low molecular weight are in stark contrast to the heterogeneity observed in animal-derived chondroitin sulfate, which comprises a mixture of high- and low-molecular-weight species with variable sulfation. The use of microbial chondroitin sulfate in the present invention is based on its demonstrated superior pharmacokinetic profile and therapeutic efficacy, including enhanced plasma concentration and charge density compared to animal-derived chondroitin sulfate. Moreover, low-molecular-weight glycosaminoglycans, such as the microbial chondroitin sulfate employed herein, have been widely recognized for their increased effectiveness in various biological activities, including anti-inflammatory and immunoaugmenting effects. In this embodiment, the microbial chondroitin sulfate also functions as a key component of a proprietary delivery system for GABA, leveraging its favorable biomaterial properties such as non-toxicity, biodegradability, and biocompatibility to enhance transdermal absorption of the active ingredient. The microbial chondroitin sulfate preferably has a sulfation profile that mimics that of human chondroitin sulfate to optimize its biological activity and interaction with GABA.

In a third embodiment, the present invention emphasizes the synergistic interactions between Gamma-Aminobutyric Acid (GABA) and microbial chondroitin sulfate within the topical cream formulation, contributing to its enhanced efficacy at both the biochemical and physiological levels. Biochemically, it is hypothesized that GABA, existing as a zwitterion at physiological pH, interacts with the negatively charged sulfate groups of the microbial chondroitin sulfate through electrostatic forces. This interaction is believed to facilitate the formation of a supramolecular complex, potentially enhancing the stability and transdermal permeability of GABA. Furthermore, the hydrophilic nature of chondroitin sulfate may aid in maintaining GABA in a hydrated state conducive to skin penetration, while the proprietary algorithm, involving vegetal propanediol, further optimizes GABA's conformation for biological activity and delivery. Physiologically, this interaction is posited to significantly enhance the topical bioavailability of GABA, allowing for more effective penetration through the stratum corneum and delivery to localized target tissues where it can modulate GABAergic signaling pathways. This enhanced delivery system, facilitated by the microbial chondroitin sulfate, aims to maximize the therapeutic benefits of GABA for anxiety management, sleep quality enhancement, and promotion of brain-related functions, while minimizing systemic exposure. Additionally, the inherent properties of microbial chondroitin sulfate, such as its hydrating, anti-inflammatory, and potentially immunoaugmenting effects, may synergistically complement the neurological actions of GABA, leading to a more comprehensive therapeutic outcome. The formation of this GABA-chondroitin sulfate complex within the proprietary delivery system represents a key aspect of this embodiment, enabling the effective topical application of GABA despite its known challenges in systemic bioavailability.

In a fourth embodiment of the present invention, the topical cream includes a unique blend of fermented probiotic microorganisms, specifically comprising Lactobacillus ferment, Streptococcus thermophilus ferment, Saccharomyces ferment, and Bifidobacterium. In contrast to many probiotic-based products that utilize live or lyophilized bacterial cultures, this embodiment features these microorganisms in a fermented form. This fermentation process yields a rich source of postbiotics, including a complex mixture of enzymes, peptides, amino acids, vitamins, and other metabolic byproducts that are distinct from the properties of the live organisms themselves. The rationale for using fermented probiotics lies in their potential to offer a gentler yet effective approach to skin health. These fermented ingredients are believed to support a balanced skin microbiome, enhance the skin's natural barrier function, reduce inflammation, and provide moisturizing benefits. By utilizing the products of fermentation, this embodiment aims to harness the beneficial molecules produced by these specific microorganisms, potentially offering improved stability and a different spectrum of skin-enhancing properties compared to formulations containing live probiotics. This approach underscores the novelty of the present invention in its utilization of fermented probiotics to complement the effects of GABA and microbial chondroitin sulfate.

In a fifth embodiment, the present invention highlights the potential synergistic and complementary interactions between Gamma-Aminobutyric Acid (GABA), microbial chondroitin sulfate, and the fermented probiotic microorganisms, which contribute to the unique health-supporting properties of the topical cream. The microbial chondroitin sulfate is believed to enhance the transdermal absorption of GABA, facilitating its delivery to target tissues where it can modulate GABAergic signaling pathways to promote calmness and relaxation. Simultaneously, the fermented probiotic microorganisms release a complex mixture of postbiotic molecules, including enzymes, peptides, and other metabolites, which can support a balanced skin microbiome, strengthen the skin barrier function, and potentially reduce local inflammation. This improved skin health may, in turn, create a more receptive environment for the absorption of GABA. Furthermore, the inherent hydrating and potential anti-inflammatory properties of microbial chondroitin sulfate may work synergistically with both GABA and the postbiotics. For instance, the soothing effects of GABA on the nervous system could be complemented by the calming effects of a balanced skin microbiome facilitated by the fermented probiotics. Additionally, the enhanced skin hydration provided by chondroitin sulfate might improve the overall efficacy of the topical delivery system for GABA and the bioavailability of the postbiotic molecules on the skin surface. This multi-faceted interaction between GABA, microbial chondroitin sulfate, and the fermented probiotics is hypothesized to provide a unique and enhanced approach to supporting overall well-being through topical application, addressing both neurological and skin-related aspects.

In a sixth embodiment of the present invention, the topical cream incorporates phosphatidylcholine as an additional ingredient. The phosphatidylcholine utilized in this embodiment is derived from sunflower and is certified as non-genetically modified (non-GMO). This ingredient serves multiple functions within the formulation, acting as an emulsifier to ensure the stability and homogeneity of the cream, a wetting agent to improve the spreadability and skin contact, a stabilizer to maintain the integrity of the product over time, a release agent to facilitate the delivery of the active ingredients to the skin, an antioxidant to protect the formulation and potentially the skin from oxidative stress, and a dispersant to ensure even distribution of the components. Biochemically, phosphatidylcholine is the most abundant phospholipid in the cell membrane, where it plays a key role in cellular signaling pathways and synaptic function. For topical application, phosphatidylcholine further contributes to the integrity of the skin's lipid barrier in the stratum corneum, supporting hydration and providing protection against external irritants. Moreover, its amphiphilic nature and ability to form liposomes may enhance the penetration of other active ingredients, such as GABA, microbial chondroitin sulfate, and the postbiotics from the fermented microorganisms, through the skin layers, thereby potentially increasing their bioavailability and efficacy. The presence of naturally occurring Phosphatidylinositol, Phosphatidylethanolamine, and essential fatty acids within sunflower may also contribute additional skin-conditioning and nourishing benefits to the formulation, further supporting overall skin health.

In a seventh embodiment, the present invention considers the potential for interaction between phosphatidylcholine and microbial chondroitin sulfate within the topical cream, drawing parallels to the observed complex formation between heparin and phosphatidylcholine. Given the structural similarities between heparin and chondroitin sulfate as sulfated glycosaminoglycans, it is hypothesized that phosphatidylcholine, particularly its quaternary ammonium group, may interact with the negatively charged sulfate moieties of the microbial chondroitin sulfate. This interaction could potentially lead to the formation of complexes within the cream matrix, altering the solubility and partitioning properties of both molecules. Inspired by the observation of heparin's paradoxical entry into the organic phase in the presence of phosphatidylcholine, it is conceivable that similar interactions within the skin's lipid environment could influence the penetration and distribution of chondroitin sulfate, potentially enhancing its interaction with skin cells or facilitating the delivery of other active ingredients like GABA. Furthermore, the formation of such complexes might affect the organization of the lipid bilayer in the stratum corneum, potentially modulating the skin barrier function and influencing transdermal absorption. While the precise nature and extent of this interaction in the present formulation require further investigation, the potential for phosphatidylcholine to interact with and modulate the behavior of microbial chondroitin sulfate within the topical cream represents a unique aspect of this embodiment that may contribute to the overall health-supporting properties of the invention. These interactions could potentially enhance the stability of the formulation, improve the delivery of active ingredients, or even directly influence cellular signaling pathways within the skin.

In an eighth embodiment of the present invention, the topical cream includes propanediol as a key component of the delivery system. This propanediol is specifically of vegetal origin, produced through a proprietary fermentation process using plant-derived glucose. It is a natural, skin-friendly, multifunctional, and innovative cosmetic ingredient 1 that serves as an emollient, providing excellent sensory characteristics to the cream. Its inclusion in the delivery system is intended to enhance the penetration and bioavailability of the other active ingredients, such as GABA and the postbiotics from the fermented microorganisms, within the skin. Propanediol's ability to act as a wetting agent and solvent may further contribute to the stability and efficacy of the overall formulation, aligning with consumer demand for safe and pure skin-friendly products.

In a ninth embodiment of the present invention, the topical cream includes dehydroacetic acid as an enhancer to improve the bioavailability of the other active compounds. Dehydroacetic acid is an organic compound known for its antimicrobial and pharmacological properties. In this formulation, dehydroacetic acid is included for its ability to enhance the skin penetration and thus the bioavailability of ingredients such as GABA, microbial chondroitin sulfate, and the postbiotics from the fermented microorganisms, potentially increasing their effectiveness. Furthermore, dehydroacetic acid is noted for being a precursor of benzodiazepines, and importantly, receptors for dehydroacetic acid exist in the brain that are stimulated through hydrophilic/hydrophobic interactions. While applied topically, the potential for dehydroacetic acid to interact with these receptors after absorption through the skin may contribute to the overall pharmacological effects of the cream, potentially complementing the actions of GABA in promoting relaxation and influencing brain function. The antimicrobial properties of dehydroacetic acid also contribute to the preservation and stability of the topical formulation.

In a tenth embodiment of the present invention, the topical cream includes benzoic acid as an additional component. Benzoic acid is an organic compound that occurs naturally in many plants and has a well-established history of medicinal use, notably as a constituent of Whitfield's ointment for the treatment of fungal skin diseases such as tinea, ringworm, and athlete's foot. Benzoic acid and its derivatives have also been historically utilized for their topical antiseptic and inhalant decongestant properties. In the context of the present invention, benzoic acid is included for its potential to enhance the bioavailability of other active ingredients within the topical formulation, possibly by modifying the skin's permeability. Additionally, its inherent antiseptic properties may contribute to maintaining the microbial integrity of the cream and could offer a synergistic benefit in supporting overall skin health alongside the fermented probiotics. While benzoic acid was also historically used as an expectorant and analgesic, its primary role in this topical embodiment is believed to be that of an enhancer and a local antiseptic.

In an eleventh embodiment of the present invention, the topical cream includes benzyl alcohol as an additional component. Benzyl alcohol is a naturally occurring alcohol produced by many plants and commonly found in fruits, teas, and various essential oils including jasmine, hyacinth, and ylang-ylang. In this formulation, benzyl alcohol functions primarily as a bacteriostatic preservative, ensuring the microbial stability of the topical cream. Additionally, benzyl alcohol is included for its known properties as a penetration enhancer, which may facilitate the absorption of other active ingredients such as GABA, microbial chondroitin sulfate, and the fermented probiotics through the skin, thereby potentially increasing their bioavailability and efficacy. Furthermore, it is recognized that benzyl alcohol can be readily oxidized to benzoic acid, which, as detailed in a previous embodiment, also possesses antiseptic properties that may contribute to the overall benefits of the topical formulation. The inclusion of benzyl alcohol thus serves a dual purpose in this embodiment, providing both preservation and potential enhancement of the active ingredients.

In a twelfth embodiment of the present invention, the topical cream includes tocopherol (Vitamin E) as an additional component. The Vitamin E utilized in this embodiment is non-GMO and derived from a natural source. Tocopherol is a well-established antioxidant, providing protection to the skin from damaging free radicals generated by various sources including UV light, cigarette smoke, and air pollution. These antioxidant properties contribute to maintaining skin health and preventing premature aging. Furthermore, Vitamin E acts as an agonist of nuclear receptors that regulate DNA expression, which are involved in a significant number of physiological functions and have been studied for their potential in preventing and treating a wide range of diseases. In the context of the present invention, tocopherol may also function as a penetration enhancer, potentially improving the bioavailability of other active ingredients such as GABA, microbial chondroitin sulfate, and the fermented probiotics by facilitating their absorption through the skin. The multifaceted benefits of tocopherol, including its antioxidant activity and potential to influence gene expression in skin cells, make it a valuable addition to this topical formulation, contributing to both skin protection and the overall efficacy of the product.

In a thirteenth embodiment of the present invention, the topical cream includes a blend of glyceryl citrate/lactate/linoleate/oleate as an additional component. This ingredient functions primarily as a moisturizer, providing a long-lasting, rich, yet non-sticky skin feel and improving the overall sensory characteristics of the topical cream. Its inclusion contributes to the emollient properties of the formulation, helping to soften and smooth the skin. Furthermore, glyceryl citrate/lactate/linoleate/oleate is known for its high compatibility with skin and mucosa, making it a well-tolerated component of the topical formulation. While its primary role is moisturizing and enhancing skin feel, maintaining optimal skin hydration and barrier function through the use of this ingredient may indirectly enhance the bioavailability and effectiveness of the other active components, such as GABA, microbial chondroitin sulfate, and the fermented probiotics, by creating a more favorable environment for their absorption and interaction with the skin. Additionally, its properties as a component used in self-emulsifying systems may contribute to the overall stability and delivery characteristics of the cream.

In a fourteenth embodiment of the present invention, the topical cream includes water as a key component of the delivery system. Water serves as the primary solvent in the formulation and plays a crucial role in solubilizing the fermented probiotic microorganisms, ensuring their proper dispersion and activity within the topical cream. The aqueous environment provided by water is essential for the stability and functionality of the fermented probiotic components, allowing them to interact effectively with the other ingredients and the skin upon application. Furthermore, water acts as the base or vehicle for the cream, providing the necessary medium for the other components to be formulated into a cosmetically acceptable and easily applicable product.

In a fifteenth embodiment of the present invention, the topical cream includes a vegetable blend comprising: Castor Oil (Ricinus communis); Hydrogenated Soybean Oil; Hydrogenated Castor Oil (Ricinus communis Seed Oil); Coconut Oil (Cocos nucifera Oil); Ouricury Wax (Arecaceae Oil); Rapeseed Oil (Brassica napus Oil); Olive Oil (Olea europaea Oil); Cottonseed Oil (Cottonseed Oil); and Stearic Acid (Stearic Acid from Palm Oil). This blend of natural plant-derived oils and waxes serves multiple crucial functions within the topical formulation. It primarily acts as an emollient and moisturizing agent, helping to soften and hydrate the skin, providing a rich and lasting skin feel. The hydrogenated oils and ouricury wax contribute occlusive properties, forming a protective layer on the skin's surface to prevent moisture loss. Coconut oil and olive oil are included for their additional skin-conditioning benefits and potential to nourish the skin with fatty acids and antioxidants. Stearic acid, derived from palm oil, acts as a thickening agent and can also contribute to the stability of the emulsion. The specific combination of these ingredients is carefully selected to provide the desired texture, spreadability, and skin feel for the topical cream, while also contributing to its overall moisturizing and protective properties.

### Example Label of the Topical Cream

The following provides an illustrative example of a label for the topical cream, demonstrating one potential way the product, formulated in accordance with one or more of the embodiments described herein, may be presented to consumers, including key information such as ingredients, usage instructions, and branding elements.

### BRAVO Microbiome Advanced Formula Cream Net Wt 2 Oz

Warning: Store in a cool, dry place. Keep out of the reach of children. If you are pregnant or lactating, consult your physician before using this product. Discontinue use if rash or irritation occurs. For external use only. Caution: Avoid contact with eyes. If contact occurs flush thoroughly with water for several minutes. Consult a healthcare professional if irritation persists. Direction for use: Apply to skin and massage until absorbed. Ingredients: Vegetable oil, propanediol, dehydroaceticacid, benzoic acid, benzyl alcohol, tocopherol (vitamin E), water, glyceryl citrate/lactate/linoleate/oleate, microbial sodium chondroitin sulfate, GABA (Gamma-Aminobutyric Acid), phosphatidylcholine, Lactobacillus ferment, Streptococcus thermophilus ferment, Saccharomyces ferment, Bifidobacterium. Silver Spring Sagl. www.bravo-europe.com

### Method of Manufacturing the Topical Cream

The following provides a description of the method for manufacturing the topical cream, utilizing the ingredients described in the preceding embodiments to produce a formulation suitable for topical application. Microbial chondroitin sulfate and probiotics (ingredients that come in powder) are dissolved in propanediol. Light heating allowed (not above 40° C. or 104 F). Then all the other ingredients are added according to the table pasted below.

Cream #1

| | | | Provided |
|---|---|---|---|
| Ingredients | Brand and supplier | Percentage | by |
| Blend of vegetal | Sonnenatural by Sonneborn | 93.47 | Kleen |
| oils: castor oil, | www.sonneborn.com | | Concepts |
| olive oil, castor | | | |
| wax, palm wax, | | | |
| coconut oil, | | | |
| hydrogenated soy | | | |
| oil | | | |
| Propanediol | Dupont-Tate and Lyle | 4.4 | Kleen |
| (vegetal source, | www.duponttateandlyle.com | | Concepts |
| organic-derived) | Other brands acceptable | | |
| | provided it is vegetal | | |
| Blend of | Sharomix 706 Plus by Acme | 0.6 | Kleen |
| dehydroacetic | Hardesty | | Concepts |
| acid and benzoic | www.acme-hardesty.com | | |
| acid in benzyl | Other brands acceptable | | |
| alcohol and water | | | |
| with 0.2% d- | | | |
| alpha-tocopherol | | | |
| (Blend of organic | | | |
| acids, free of | | | |
| parabens, | | | |
| formaldehyde | | | |
| donors, halogens | | | |
| and | | | |
| isothiazolinones) | | | |
| Vitamin E | — | 0.5 | Kleen |
| | | | Concepts |
| Glyceryl | Imwitor 375 by Oleochem | 0.5 | Kleen |
| citrate/lactate/ | www.ioioleo.de | | Concepts |
| linoleate/oleate | Other brands acceptable | | |
| (natural | | | |
| emulsifiers) | | | |
| Liquid sunflower | — | 0.4 | Kleen |
| lecithin (soy free, | | | Concepts |
| non-GMO) | | | |
| Microbial | Mythocondro by Gnosis | 0.1 | Dolce Vita |
| chondroitin | www.gnosisbylesaffre.com | | LLC |
| sulfate (powder) | | | |
| Probiotics | Silver Spring Sagl | 0.03 | Dolce Vita |
| (powder) | www.bravo-europe.com | | LLC |

### Observed Effects of the Topical Cream in Human Subjects

To evaluate the effects of the topical cream formulated according to the methods and compositions described herein, a study was conducted involving a number of volunteer subjects with varying demographic profiles. The subjects were instructed to apply the cream to the back of the ears and rub it gently for approximately 30 seconds prior to bedtime. The effects reported by the subjects were related to sleep initiation, sleep maintenance, sleep quality, daytime alertness, mood, and cognitive function. The observed effects are detailed below.

The following effects were reported by one or more of the volunteer subjects: Ease in initiating sleep and a decrease in the time taken to fall asleep; Elimination or significant reduction of nocturnal awakenings; Ease in falling back asleep in case of sporadic nocturnal awakenings; Improvement in the overall quality and efficiency of sleep; Elimination or significant reduction of daytime somnolence, if present; Feeling of increased energy upon awakening; Improvement of mood during the day. Improvement of focus and attention (for example, while studying); and Increase in the number and quality of dreams.

A majority of the subjects reported significant effects on the quality and efficiency of sleep after the first application of the cream. In some instances, the effects on focus, attention, or mood were observed after a few days of application. The effects appeared to persist throughout the day, and a majority of subjects reported no need for reapplication of the cream during the day. To date, no adverse events or side effects have been communicated by any of the subjects who participated in the study.

The following testimonials are representative of observations describing consistent responses to the application of the cream among volunteer subjects.

Subject B. L., 72-year-old female: This subject reported positive changes in sleep rhythm and quality from the first night of cream application. Prior to using the cream, the subject typically experienced sleep onset around 10 PM but would wake at least once during the night with difficulty returning to sleep, sometimes remaining awake until morning. Following the application of the cream, the subject reported easier sleep onset from the first application, and in some instances, no nocturnal awakenings occurred. On occasions of waking during the night (around 3 or 4 AM), the subject reported falling back asleep within a few minutes without difficulty, experiencing a sustained and restful sleep until the following morning.

Subject L. T., 72-year-old female: This subject typically experienced late sleep onset and sleep frequently disturbed by nightmares. Since using the cream, the subject reported a decreased frequency of nightmares, with some nights being free of nightmares, and an overall more relaxed sleep. The subject also reported waking up feeling more serene and rested.

Subject M. S., 63-year-old female: This subject typically experienced multiple nocturnal awakenings, often associated with anxiety and anguish, making it very difficult to fall back asleep. Following the first night of using the cream, and continuing thereafter, the subject reported a significant reduction in nocturnal awakenings, and when they did occur, the associated anxiety was diminished, facilitating easier return to sleep. The subject also reported positive changes in mood upon waking, noting a better mood that often persisted throughout the day.

Subject F. R., 29-year-old male: This subject reported a much easier sleep onset, with the time taken to fall asleep decreasing from at least 2-3 hours to a maximum of half an hour after the first night of cream application. After a few weeks of use, the subject reported waking up in the morning feeling full of energy.

Subject L. G., 28-year-old female: This subject reported noticing improved concentration and increased energy upon waking after using the cream for approximately four to five days, describing the sensation as similar to that experienced after drinking a strong coffee.

Subject S. P., 52-year-old female: This subject, who typically experienced good sleep patterns, reported a notable and interesting variation in the type and intensity of dreams from the first night of cream application. The dreams were described as much more detailed than usual, more numerous, and more intense. Notably, the subject reported experiencing olfactory sensations during dreams, a phenomenon not previously experienced. Due to the quantity and richness of detail in the dreams, the subject reported keeping a pencil and paper by the bedside to record them in the morning, documenting up to six different dreams with significant detail per night. The subject found this product to be of significant interest with many potential fields of use.

To provide objective evidence of the effects of the topical cream, instrumental measurements were conducted on a volunteer subject utilizing a Muse EEG neurofeedback system. The subject participated in unguided neurofeedback training sessions both before and approximately 30 minutes after the application of the cream. Quantitative data regarding the subject's mental state and stillness were recorded during these sessions.

Prior to the application of the cream, the subject performed a 5-minute unguided neurofeedback training session denominated “Heart Session Healing Drum” as provided by the Muse EEG system. Approximately 30 minutes after the application of the cream, the subject performed another identical 5-minute session. Both sessions were conducted in a dimly illuminated, quiet room, free from potential distractions, with the subject maintaining a Seiza position. The results of these sessions were recorded on the subject's smartphone and exported directly as screenshots without modification.

Following the application of the cream, the overall percentage of time spent in a calm state increased from 41% to 66%. Specifically, the duration of the calm state increased from 125 seconds to 199 seconds, representing a 60% increase. Concurrently, the duration of the neutral state decreased from 173 seconds to 100 seconds. The subject's heart rate did not exhibit a significant change between the two sessions.

These results indicate an association between the application of the cream and a significant increase in mental calmness, a phenomenon consistent with the known effects of Gamma-Aminobutyric Acid (GABA).

Further observations suggest that these results are independent of the specific type of neurofeedback training session utilized within the Muse app. For example, approximately 24 hours after the observation described above, the subject performed two additional 5-minute unguided sessions, this time using a program denominated “Mind Session Desert.”

Superimposable results were observed, with an increase in calmness following the application of the cream. The time spent in a calm state increased from 75 seconds to 120 seconds, representing a 60% increase, despite the baseline percentage of time spent in a calm state being lower during these sessions compared to the previous day. This suggests that the increase in calmness may be independent of the initial mental state.

Consistent with these findings, stillness, as measured by the accelerometer of the Muse system, increased from 43% to 85% of the total session time during another 5-minute unguided neurofeedback training session denominated “Heart Session Healing Drum” following the application of the cream. More specifically, the duration of the relaxed state, as indicated by the stillness parameter, increased from 126 seconds to 255 seconds.

### SPECIFIC USES

The topical cream disclosed in the present invention, characterized by its unique combination of ingredients and demonstrated effects, is intended for the following specific uses to support health and well-being.

The topical cream described herein is particularly suited for a range of specific applications aimed at supporting overall well-being. These applications include aiding in the management of anxiety and stress, enhancing sleep quality by facilitating sleep onset, reducing nocturnal disturbances, and improving the overall quality and efficiency of sleep. The cream is also indicated for the promotion of cognitive functions such as focus and attention. Furthermore, the formulation has been observed to provide significant relief from joint pain, muscle pain, back pain, and post-traumatic pain, such as that resulting from sprains and other musculoskeletal injuries. These effects are believed to be mediated through the synergistic actions of the cream's components, including the modulation of GABAergic pathways and the potential anti-inflammatory and soothing properties of ingredients like microbial chondroitin sulfate and the fermented probiotics.

What is claimed is:

1. A topical cream comprising: Gamma-Aminobutyric Acid (GABA); Microbial chondroitin sulfate; A blend of fermented probiotic microorganisms including Lactobacillus ferment, Streptococcus thermophilus ferment, Saccharomyces ferment, and Bifidobacterium; Phosphatidylcholine; Propanediol; Dehydroacetic acid; Benzoic acid; Benzyl alcohol; Tocopherol; Glyceryl citrate/lactate/linoleate/oleate; Water; and A vegetable blend comprising Castor Oil, Hydrogenated Soybean Oil, Hydrogenated Castor Oil, Coconut Oil, Ouricury Wax, Rapeseed Oil, Olive Oil, Cottonseed Oil, and Stearic Acid.

2. The topical cream of claim 1, wherein the microbial chondroitin sulfate enhances the transdermal absorption of Gamma-Aminobutyric Acid (GABA).

3. The topical cream of claim 1 for use in improving sleep quality, managing anxiety, promoting focus and attention, and/or alleviating joint pain, muscle pain, and post-traumatic pain.

4. The topical cream of claim 1, wherein the fermented probiotic microorganisms are present in a form that yields postbiotic compounds beneficial for the skin.

5. A method of improving sleep quality, managing anxiety, promoting focus and attention, and/or alleviating joint pain, muscle pain, and post-traumatic pain, comprising topically administering the cream of claim 1 to a subject in need thereof.
